(19) United States
(12) Patent Application Publication
(10) Pub. No.: US 2023/0149669 A1
(43) Pub. Date: May 18, 2023

(54) GUIDEWIRE GUIDE CONFIGURATIONS FOR IMPLANTABLE MEDICAL DEVICES

(71) Applicant: TC1 LLC, St. Paul, MN (US)

(72) Inventors: Michael R. Butler, Dublin, CA (US); Phyllis Yuen, Fremont, CA (US); Keif Fitzgerald, San Jose, CA (US); Victoria M. Gong, Sunnyvale, CA (US)

(21) Appl. No.: 18/048,652

(22) Filed: Oct. 21, 2022

Related U.S. Application Data
(60) Provisional application No. 63/280,803, filed on Nov. 18, 2021.

(51) Int. Cl. A61M 25/09; A61M 60/13; A61M 25/10

(52) U.S. Cl. CPC A61M 25/09; A61M 60/13; A61M 25/10; A61M 2025/09133; A61M 2025/09008; A61M 2025/1093

(57) ABSTRACT

A catheter assembly for use in a percutaneous heart pump is provided. The catheter assembly may include a GWG that includes a hypotube and a sleeve section coupled to a distal end of the hypotube. The sleeve section is configured to extend across a distal septum of the percutaneous heart pump when the GWG is inserted into the percutaneous heart pump, and facilitate reducing deformation of the distal septum while the sleeve section extends across the distal septum.

### CROSS-REFERENCE TO RELATED APPLICATIONS

This application claims priority to U.S. Provisional Patent Application No. 63/280,803, filed on Nov. 18, 2021, titled Guidewire Guide Configurations for Implantable Medical Devices, the entire contents of which are hereby incorporated herein by reference.

### FIELD OF THE INVENTION

The present disclosure relates generally to implantable medical devices, and more specifically, relates to guidewire guides for use in implantable medical devices.

### BACKGROUND

Heart disease is a major health problem that has a high mortality rate. Physicians increasingly use mechanical circulatory support systems for treating heart failure. The treatment of acute heart failure requires a device that can provide support to the patient quickly. Physicians desire treatment options that can be deployed quickly and minimally-invasively.

A Percutaneous Heart Pump (PHP) system is one example of a ventricular assist device that may be used during high-risk percutaneous coronary interventions (PCI) performed electively or urgently in hemodynamically stable patients with severe coronary artery disease, when a heart team, including a cardiac surgeon, has determined high-risk PCI is the appropriate therapeutic option. Use of the PHP system in these patients may prevent hemodynamic instability, which can result from repeat episodes of reversible myocardial ischemia that occur during planned temporary coronary occlusions and may reduce pre-and post-procedural adverse events. PHP systems may also be used to treat cardiogenic shock in certain circumstances.

In at least some embodiments, the PHP system includes a distal septum to prevent blood from entering a fluid lumen of a catheter of the PHP system. In such embodiments, a guidewire guide (GWG) may extend through the distal septum for a period of time (e.g., while the PHP system is being stored), which may impact the sealing capabilities of the distal septum. Accordingly, it would be desirable to provide a GWG that facilitates reducing impacts on the sealing capabilities of the distal septum.

### BRIEF SUMMARY OF THE DISCLOSURE

The present disclosure is directed to a guidewire guide (GWG) for use in a percutaneous heart pump. The GWG includes a hypotube and a sleeve section. The sleeve section is configured to extend across a distal septum of the percutaneous heart pump when the GWG is inserted into the percutaneous heart pump. The sleeve section also facilitates reducing deformation of the distal septum while the sleeve section extends across the distal septum.

The present disclosure is also directed to a catheter assembly for use in a percutaneous heart pump. The catheter assembly includes an impeller assembly that includes an impeller, an impeller tip positioned distal of the impeller, and a distal septum positioned between the impeller and the impeller tip. The catheter assembly also includes a flexible atraumatic tip (FAT) positioned distal of the impeller assembly, and a guidewire guide (GWG) coupled between the impeller assembly and the flexible atraumatic tip, wherein a proximal end of the guidewire guide is positioned distal of the distal septum.

The present disclosure is further directed to a catheter assembly for use in a percutaneous heart pump. The catheter assembly includes a guidewire guide (GWG) that includes a hypotube and a sleeve section coupled to a distal end of the hypotube. The sleeve section is configured to extend across a distal septum of the percutaneous heart pump when the GWG is inserted into the percutaneous heart pump. The sleeve section also facilitates reducing deformation of the distal septum while the sleeve section extends across the distal septum.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 illustrates one embodiment of a catheter pump configured for percutaneous application and operation.

FIG. 2 is a plan view of one embodiment of a catheter adapted to be used with the catheter pump of FIG. 1.

FIG. 3 shows a distal portion of the catheter system similar to that of FIG. 2 in position within the anatomy.

FIG. 4 is a schematic view of a catheter assembly and a drive assembly.

FIG. 4A is an enlarged view of a priming apparatus shown in FIG. 4.

FIG. 5 is a perspective view of a motor assembly as the drive assembly is being coupled to a driven assembly.

FIG. 6 is a plan view of the motor assembly once the drive assembly has been coupled and secured to a driven assembly.

FIG. 7 is a perspective view of the motor assembly of FIG. 6, with various components removed for ease of illustration.

FIG. 8 is a plan view of the motor assembly that illustrates a motor, a drive magnet, and a driven magnet, with various components removed for ease of illustration.

FIG. 9 is a perspective view of a first securement device configured to secure the drive assembly to the driven assembly, with various components removed for ease of illustration.

FIGS. 10A-10C are perspective views of a second securement device configured to secure the drive assembly to the driven assembly.

FIG. 11 illustrates a side schematic view of a motor assembly according to another embodiment.

FIGS. 12A and 12B illustrate side schematic views of a motor assembly according to yet another embodiment.

FIG. 13 is a side view of a distal tip member disposed at a distal end of the catheter assembly, according to one embodiment.

FIG. 14 is a side cross-sectional view of a distal tip member disposed at a distal end of the catheter assembly, according to another embodiment.

FIG. 15 is a side cross-sectional view of a catheter assembly including an embodiment of a guidewire guide (GWG), according to one embodiment.

FIG. 16 is a perspective cross-sectional view of a catheter assembly including an alternative embodiment of a GWG, according to one embodiment.

FIG. 17 illustrates various embodiments of a distal portion of a GWG tube.

FIG. 18 illustrates an enlarged view of a portion of a catheter assembly including one embodiment of the various embodiments of the distal portion of the GWG tube shown in FIG. 17 extending across a distal septum.

Corresponding reference characters indicate corresponding parts throughout the several views of the drawings.

### DETAILED DESCRIPTION OF THE DISCLOSURE

The disclosure provides systems and methods for guidewire guides (GWGs) for use in a medical system. In various embodiments disclosed herein, a GWG may be configured to receive a guidewire therethrough. A clinician may maneuver the guidewire to the heart through the patient's vasculature. The clinician may then advance a distal portion of a catheter assembly over the guidewire, using the GWG, to position the distal portion (e.g., including an impeller) in a chamber of the heart. In some embodiments, the GWG may include a central lumen formed along the length of the catheter assembly.

Additional embodiments of this disclosure are directed to apparatuses for inducing motion of a fluid relative to the apparatus. For example, an operative device, such as an impeller, may be coupled at a distal portion of the apparatus. In particular, various embodiments disclosed herein generally relate to various configurations for a motor (also referred to herein as a “motor assembly”) adapted to drive an impeller at a distal end of a catheter pump (e.g., a percutaneous heart pump). The motor assembly may be disposed outside the patient in some embodiments. In other embodiments, the motor assembly may be miniaturized and sized to be inserted within the body.

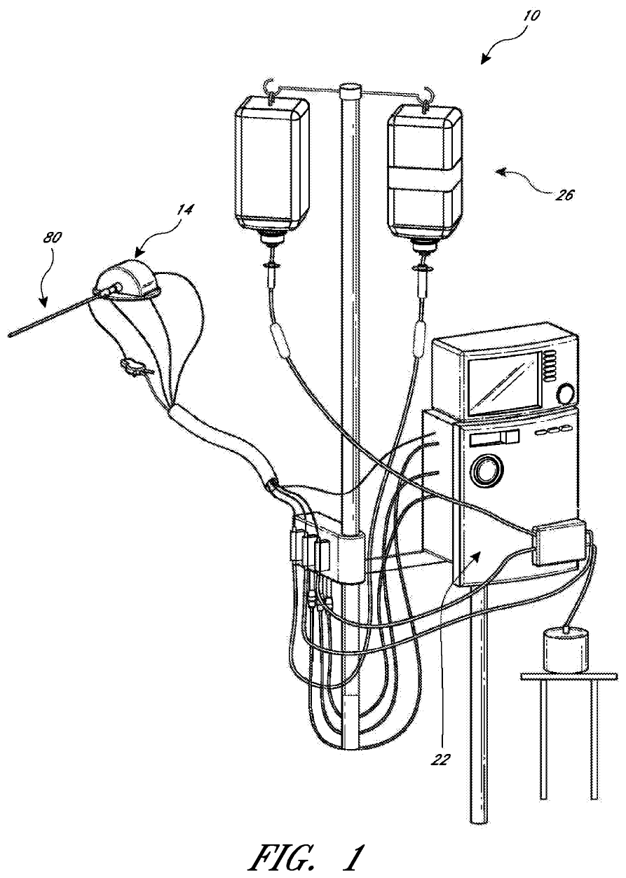

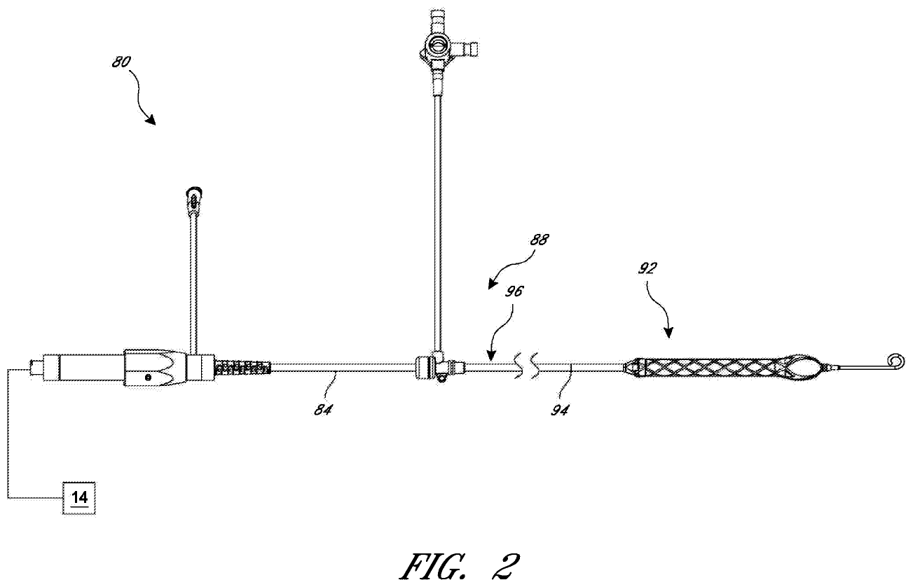

FIG. 1 illustrates aspects of a catheter pump 10 that may provide high performance flow rates. Catheter pump 10 includes a motor 14 driven by a controller 22. Controller 22 directs the operation of motor 14 and an infusion system 26 that supplies a flow of infusate in catheter pump 10. A catheter assembly 80 that may be coupled to motor 14 houses an impeller within a distal portion thereof. In various embodiments, the impeller is rotated remotely by motor 14 when catheter pump 10 is operating. For example, motor 14 may be disposed outside the patient. In some embodiments, motor 14 is separate from controller 22 (e.g., to be placed closer to the patient). In other embodiments, motor 14 is part of controller 22. In still other embodiments, motor 14 is miniaturized to be insertable into the patient. Such embodiments allow the drive shaft to be much shorter (e.g., shorter than the distance from the aortic valve to the aortic arch (about 5 cm or less)). Some examples of miniaturized motors, catheter pumps, and related components and methods are discussed in U.S. Pat. Nos. 5,964,694, 6,007,478, 6,178,922, and 6,176,848, all of which are incorporated herein by reference for all purposes in their entirety. Various embodiments of motor 14 are disclosed herein, including embodiments having separate drive and driven assemblies to enable the use of a GWG passing through catheter pump 10. As explained herein, a GWG may facilitate passing a guidewire through catheter pump 10 for percutaneous delivery of catheter pump 10's operative device to a patient's heart.

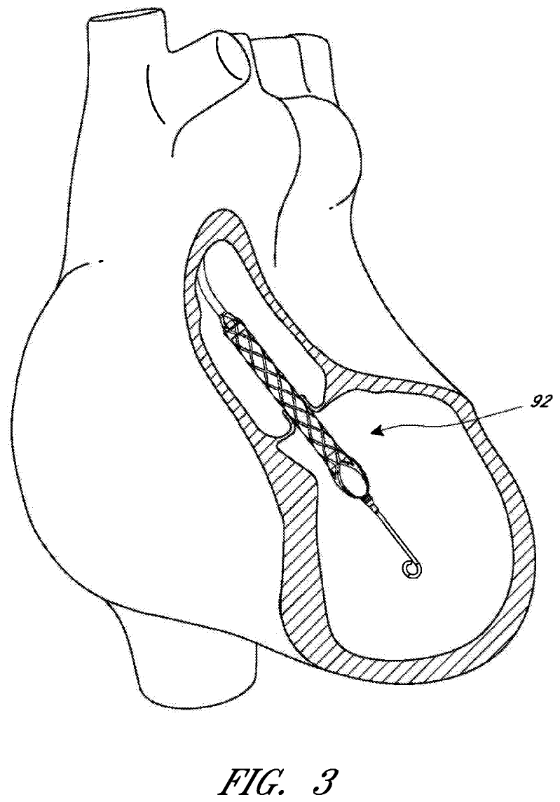

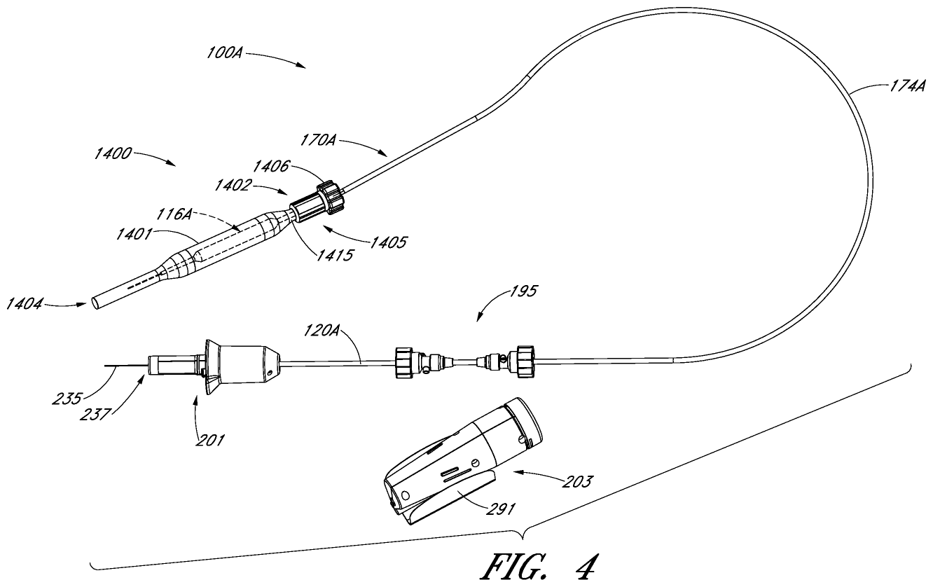

FIG. 2 illustrates features that facilitate small blood vessel percutaneous delivery and high performance, including up to and in some cases exceeding normal cardiac output in all phases of the cardiac cycle. In particular, catheter assembly 80 includes a catheter body 84 and a sheath assembly 88. Catheter assembly 80 is also coupled to motor 14, as described above. Impeller assembly 92 is coupled to a distal end of catheter body 84. Impeller assembly 92 is expandable and collapsible. In the collapsed state, the distal end of catheter assembly 80 may be advanced to the heart, for example, through an artery. In the expanded state, impeller assembly 92 is able to pump blood at high flow rates. FIGS. 2 and 3 illustrate the expanded state of impeller assembly 92. The collapsed state may be provided by advancing a distal end 94 of an elongate body 96 distally over impeller assembly 92 to cause impeller assembly 92 to collapse. This provides an outer profile throughout catheter assembly 80 that is of small diameter, for example, to a catheter size of about 12.5 FR in various arrangements.

In some embodiments, impeller assembly 92 includes a self-expanding material that facilitates expansion. Catheter body 84 is preferably a polymeric body that has high flexibility. When impeller assembly 92 is collapsed, as discussed above, high forces are applied to impeller assembly 92. These forces are concentrated at a connection zone, where impeller assembly 92 and catheter body 84 are coupled together. These high forces, if not carefully managed, may result in damage to catheter assembly 80 and, in some cases, render an impeller within impeller assembly 92 inoperable. Robust mechanical interface are provided to assure high performance.

The mechanical components rotatably supporting the impeller within impeller assembly 92 permit high rotational speeds while controlling heat and particle generation that may come with high speeds. Infusion system 26 (as shown in FIG. 1) delivers a cooling and lubricating solution to the distal portion of catheter assembly 80 for these purposes. However, the space for delivery of this fluid is extremely limited. Some of the space is also used for return of the infusate. Providing secure connection and reliable routing of infusate into and out of catheter assembly 80 is critical and challenging in view of the small profile of catheter body 84.

When activated, catheter pump 10 (shown in FIG. 1) may effectively increase the flow of blood out of the heart and through the patient's vascular system. In various embodiments, catheter pump 10 may be configured to produce a maximum flow rate (e.g., low mm Hg) of greater than 4 Lpm, greater than 4.5 Lpm, greater than 5 Lpm, greater than 5.5 Lpm, greater than 6 Lpm, greater than 6.5 Lpm, greater than 7 Lpm, greater than 7.5 Lpm, greater than 8 Lpm, greater than 9 Lpm, or greater than 10 Lpm. In various embodiments, catheter pump 10 may be configured to produce an average flow rate at 62 mmHg of greater than 2 Lpm, greater than 2.5 Lpm, greater than 3 Lpm, greater than 3.5 Lpm, greater than 4 Lpm, greater than 4.25 Lpm, greater than 4.5 Lpm, greater than 5 Lpm, greater than 5.5 Lpm, or greater than 6 Lpm.

Various aspects of the pump and associated components are similar to those disclosed in U.S. Pat. Nos. 7,393,181, 8,376,707, 7,841,976, 7,022,100, 7,998,054, 8,721,517, 9,358,329, 9,446,179, 9,872,947, and 10,449,279 and in U.S. Patent Publication Nos. 2011/0004046, 2012/0178986, 2012/0172655, 2012/0178985, and 2012/0004495, all of which are incorporated herein by reference for all purposes in their entirety. In addition, this disclosure incorporates by reference in its entirety and for all purposes the subject matter disclosed in the following filed application: Application No. 61/780,656, entitled “FLUID HANDLING SYSTEM,” filed on Mar. 13, 2013.

FIG. 3 illustrates one use of catheter pump 10 (shown in FIG. 1). A distal portion of catheter pump 10, which may include an impeller assembly 92, is placed in the left ventricle (“LV”) of the heart to pump blood from the LV into the aorta. Catheter pump 10 may be used in this way to treat patients with a wide range of conditions, including cardiogenic shock, myocardial infarction, and other cardiac conditions, and also to support a patient during a procedure, such as percutaneous coronary intervention. One convenient manner of placement of the distal portion of catheter pump 10 in the heart is by percutaneous access and delivery using the Seldinger technique or other methods familiar to cardiologists. Various guide features disclosed herein enable catheter pump 10 to be advanced over a guidewire to the heart. These approaches enable catheter pump 10 to be used in emergency medicine, a catheter lab, and in other non-surgical settings. Modifications may also enable catheter pump 10 to support the right side of the heart. Example modifications that may be used for right side support include providing delivery features and/or shaping a distal portion that is to be placed through at least one heart valve from the venous side, such as discussed in U.S. Pat. Nos. 6,544,216 and 7,070,555 and in U.S. Patent Publication No. 2012/0203056, all of which are incorporated herein by reference for all purposes in their entirety.

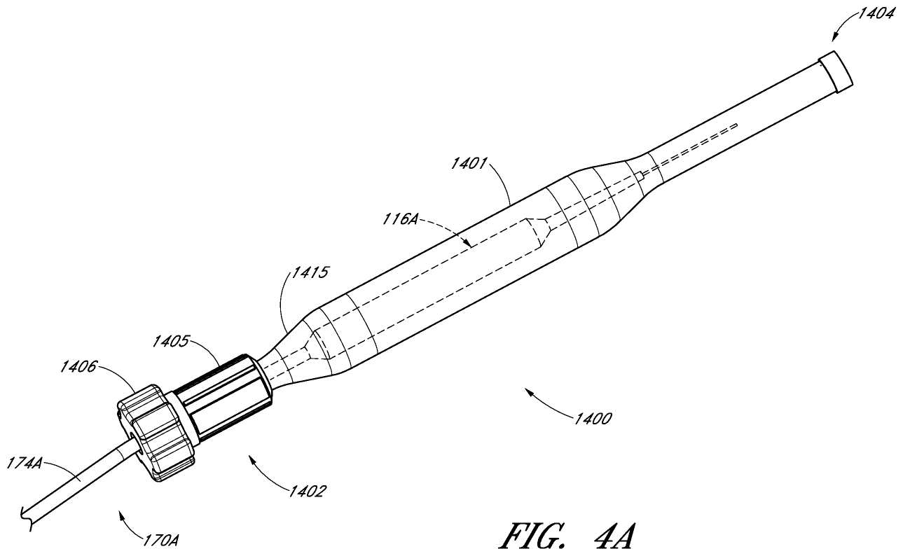

FIG. 4 illustrates another example of a catheter assembly, such as a catheter assembly 100A (similar to catheter assembly 80 shown in FIG. 2). Embodiments of catheter pumps, such as catheter pump 10 (shown in FIG. 1) of this disclosure may be configured with a motor, such as motor 14 (shown in FIG. 1) that is capable of coupling to (and in some arrangements optionally decoupling from) catheter assembly 100A. This arrangement provides a number of advantages over a non-disconnectable housing. For example, access may be provided to a proximal end 1402 of catheter assembly 100A prior to or during use. In one embodiment, catheter assembly 100A is delivered over a guidewire 235. In some embodiments, guidewire 235 may be conveniently extended through the entire length of catheter assembly 100A and out of a proximal portion thereof that is completely enclosed in a coupled configuration. For this approach, connection of the proximal portion of catheter assembly 100A to a motor housing of motor 14 may be completed after guidewire 235 has been used to guide the operative device of catheter assembly 100A to a desired location within the patient (e.g., to a chamber of the patient's heart). In one embodiment, the connection between the motor housing and catheter assembly 100A is configured to be permanent, such that catheter assembly 100A, the motor housing, and motor 14 are disposable components. However, in other implementations, the coupling between the motor housing and catheter assembly 100A is disengageable, such that motor 14 and motor housing may be decoupled from catheter assembly 100A after use. In such embodiments, catheter assembly 100A distal of motor 14 may be disposable, and motor 14 and the motor housing may be re-usable. In other embodiments, as explained in more detail below, guidewire 235 may be inserted through other types of guide features to guide catheter pump 10 to the heart. For example, in other embodiments, there may be no central lumen extending from proximal end 1402 to the distal end of catheter assembly 100A. Rather, guidewire 235 may be inserted along the side of catheter assembly 100A or along a short central lumen or a removable lumen.

Moving from the distal end of catheter assembly 100A to proximal end 1402, a priming apparatus 1400 may be disposed over an impeller assembly 116A, such as impeller assembly 92 (shown in FIGS. 2 and 3). As explained above, impeller assembly 116A may include an expandable cannula or housing and an impeller with one or more blades. As the impeller rotates, blood may be pumped proximally (or distally in some implementations) to function as a cardiac assist device.

FIG. 4 also illustrates one example of priming apparatus 1400 disposed over impeller assembly 116A near a distal end 170A of an elongate body 174A. FIG. 4A is an enlarged view of priming apparatus 1400 shown in FIG. 4. Priming apparatus 1400 may be used in connection with a procedure to expel air from impeller assembly 116A (e.g., any air that is trapped within the housing or that remains within elongate body 174A near distal end 170A). For example, a priming procedure may be performed before catheter pump 10 is inserted into the patient's vascular system, so that air bubbles are not allowed to enter and/or injure the patient. Priming apparatus 1400 may include a primer housing 1401 configured to be disposed around both elongate body 174A and impeller assembly 116A. A sealing cap 1406 may be applied to proximal end 1402 of primer housing 1401 to substantially seal priming apparatus 1400 for priming (i.e., so that air does not proximally enter elongate body 174A and also so that priming fluid does not flow out of proximal end 1402 of housing 1401). Sealing cap 1406 may be coupled to primer housing 1401 in any way known to a skilled artisan. However, in some embodiments, sealing cap 1406 is threaded onto primer housing 1401 by way of a threaded connector 1405 located at proximal end 1402 of primer housing 1401. Sealing cap 1406 may include a sealing recess disposed at the distal end of sealing cap 1406. The sealing recess may be configured to enable elongate body 174A to pass through sealing cap 1406.

The priming procedure may proceed by introducing fluid into sealed priming apparatus 1400 to expel air from impeller assembly 116A and elongate body 174A. Fluid may be introduced into priming apparatus 1400 in a variety of ways. For example, fluid may be introduced distally through elongate body 174A into priming apparatus 1400. In other embodiments, an inlet, such as a luer, may optionally be formed on a side of primer housing 1401 to enable introduction of fluid into priming apparatus 1400.

A gas permeable membrane may be disposed on a distal end 1404 of primer housing 1401. The gas permeable membrane may permit air to escape from primer housing 1401 during priming. Further, priming apparatus 1400 may advantageously be configured to collapse an expandable portion of catheter assembly 100A. Primer housing 1401 may include a funnel 1415 where the inner diameter of the housing decreases from distal to proximal. Funnel 1415 may be gently curved such that relative proximal movement of an impeller housing causes the impeller housing to be collapsed by funnel 1415. During or after the impeller housing has been fully collapsed, distal end 170A of elongate body 174A may be moved distally relative to the collapsed impeller housing. After the impeller housing is fully collapsed and retracted into elongate body 174A of a sheath assembly (such as sheath assembly 88 shown in FIG. 2), catheter assembly 100A may be removed from priming apparatus 1400 before a percutaneous heart procedure is performed (e.g., before catheter pump 10 is activated to pump blood). The embodiments disclosed herein may be implemented such that the total time for infusing the system is minimized or reduced. For example, in some embodiments, the time to fully infuse the system can be about six minutes or less. In other embodiments, the time to infuse may be about three minutes or less. In yet other embodiments, the total time to infuse the system may be about 45 seconds or less. It should be appreciated that lower times to infuse may be advantageous for use with cardiovascular patients.

Continue referencing to FIG. 4, elongate body 174A extends proximally from impeller assembly 116A to an infusate device 195 configured to enable for infusate to enter catheter assembly 100A and for waste fluid to leave catheter assembly 100A. A catheter body 120A (which also passes through elongate body 174A) may extend proximally and be coupled to a driven assembly 201. Driven assembly 201 may be configured to receive torque applied by a drive assembly 203, which is shown as being decoupled from driven assembly 201 and catheter assembly 100A in FIG. 4. Although not shown in FIG. 4, a drive shaft may extend from driven assembly 201 through catheter body 120A to couple to an impeller shaft at or proximal to impeller assembly 116A. Catheter body 120A may pass within elongate body 174A such that elongate body 174A may axially translate relative to catheter body 120A.

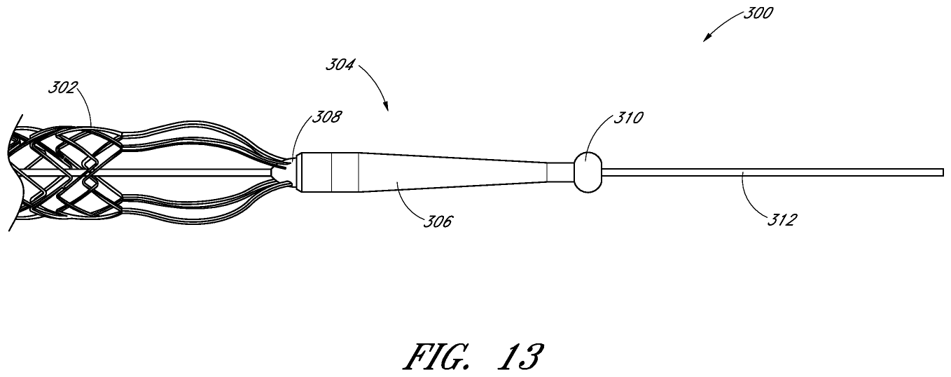

In addition, FIG. 4 illustrates guidewire 235 extending from a proximal guidewire opening 237 in driven assembly 201. Before inserting catheter assembly 100A into a patient, a clinician may insert guidewire 235 through the patient's vascular system to the heart to prepare a path for the operative device (e.g., impeller assembly 116A) to the heart. In some embodiments, catheter assembly 100A may include a guidewire guide (GWG) tube, such as GWG tube 312 shown in FIG. 13, passing through a central internal lumen of catheter assembly 100A from proximal guidewire opening 237. The GWG tube may be pre-installed in catheter assembly 100A to provide the clinician with a preformed pathway along which to insert guidewire 235. Thus, in the embodiment of FIG. 4, guidewire 235 may be advanced through a central lumen extending through the length of catheter assembly 100A. Other embodiments may include different types of guide features, as explained herein.

In one approach, guidewire 235 is first placed in a conventional way, e.g., through a needle into a peripheral blood vessel, and along the path between that blood vessel and the heart and into a heart chamber (e.g., into the left ventricle). Thereafter, a distal end opening of catheter assembly 100A and/or the GWG tube may be advanced over a proximal end of guidewire 235 to enable delivery to catheter assembly 100A. After the proximal end of guidewire 235 is urged proximally within catheter assembly 100A and emerges from proximal guidewire opening 237 and/or the GWG tube, catheter assembly 100A may be advanced into the patient. In one method, guidewire 235 is withdrawn proximally while holding catheter assembly 100A.

Alternatively, the clinician may insert guidewire 235 through proximal guidewire opening 237 and urge guidewire 235 along the GWG tube until guidewire 235 extends from a distal guidewire opening (not shown) in the distal end of catheter assembly 100A. The clinician may continue urging guidewire 235 through the patient's vascular system until the distal end of guidewire 235 is positioned in the desired chamber of the patient's heart. As shown in FIG. 4, a proximal end portion of guidewire 235 may extend from proximal guidewire opening 237. Once the distal end of guidewire 235 is positioned in the heart, the clinician may maneuver impeller assembly 116A over guidewire 235 until impeller assembly 116A reaches the distal end of guidewire 235 in the heart. The clinician may remove guidewire 235 and the GWG tube. In some embodiments, the GWG tube may also be removed before or after guidewire 235 is removed. Other embodiments for inserting guidewire 235 through different types of guide features are explained in more detail below.

After removing at least guidewire 235, the clinician can activate a motor, such as motor 14 (shown in FIG. 2) to rotate the impeller and begin operation of catheter pump 10. One problem that arises when using guidewire 235 to guide the operative device to the heart is that a central lumen or tube (e.g., a GWG) is typically formed to provide a path for guidewire 235. In some embodiments, it may be inconvenient or inoperable to provide a motor or drive assembly 203 having a lumen through which guidewire 235 may pass through. Moreover, in some implementations, it may be desirable to provide motor or drive assembly 203 separate from catheter assembly 100A (e.g., for manufacturing or economic purposes). Thus, it may be advantageous to provide a means to couple drive assembly 203 to driven assembly 201, while enabling the use of a GWG through which guidewire 235 may be passed. Preferably, drive assembly 203 may be securely coupled to driven assembly 201 such that vibratory, axial, or other external forces do not decouple drive assembly 203 from driven assembly 201 during operation. Moreover, the coupling should preferably enable the motor to operate effectively so that the drive shaft is rotated at the desired speed and with the desired torque.

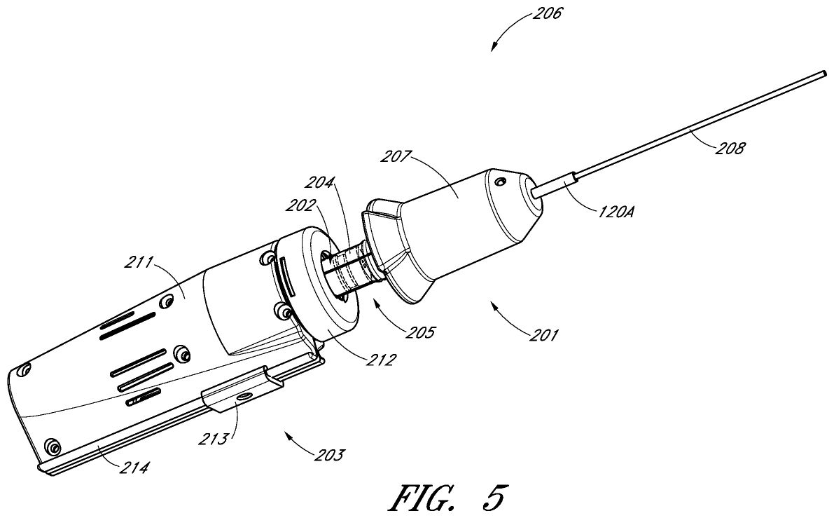

FIG. 5 illustrates one embodiment of a motor assembly 206 as driven assembly 201 is being coupled to drive assembly 203. Driven assembly 201 may include a flow diverter 205 and a flow diverter housing 207 that houses flow diverter 205. Flow diverter 205 may include a plurality of internal cavities, passages, and channels that are configured to route fluid to and from the patient during a medical procedure. As discussed below, an infusate may be directed into flow diverter 205 from a source of the infusate. The infusate is a fluid that flows into catheter body 120A to provide useful benefits, such as cooling moving parts and keeping blood from entering certain parts of catheter assembly 100A. The infusate is diverted distally by flow channels in flow diverter 205. Some of the infusate that flows distally is re-routed back through catheter body 120A and may be diverted out of catheter assembly 100A by flow diverter 205. In various embodiments, a driven magnet 204 may be disposed within flow diverter 205. For example, driven magnet 204 may be journaled for rotation in a proximal portion of flow diverter housing 207. The proximal portion may project proximally of a proximal face of a distal portion of flow diverter housing 207. In other embodiments, driven magnet 204 may be disposed outside flow diverter 205. Driven magnet 204 may be configured to rotate freely relative to flow diverter 205 and/or flow diverter housing 207. Catheter body 120A may extend from a distal end of flow diverter housing 207. Further, a drive shaft 208 may pass through catheter body 120A from a proximal end of flow diverter housing 207 to distal end 170A of elongate body 174A (both shown in FIGS. 4 and 4A). Drive shaft 208 may be configured to drive the impeller located at the distal end of catheter assembly 100A. In some embodiments, a distal end of drive shaft 208 may be coupled to an impeller shaft, which rotates the impeller.

Drive assembly 203 may include a drive housing or a motor housing 211 having an opening 202 in a cap 212 of motor housing 211. Motor housing 211 may also have a sliding member 213, which may be configured to couple to the patient's body by way of, for example, connector 291 (shown in FIG. 4) coupled to an adhesive or bandage on the patient's body. Because the motor and motor housing 211 may have a relatively high mass, it can be important to ensure that motor housing 211 is stably supported. In one embodiment, therefore, motor housing 211 may be supported by the patient's body by way of sliding member 213 and connector 291. Sliding member 213 can slide along a track 214 located on a portion of motor housing 211, such that relative motion between motor assembly 206 and the patient does not decouple sliding member 213 from the patient's body. Sliding member 213 and connector 291 may therefore be configured to provide a structural interface between motor housing 206 and a platform for supporting motor housing 211. As explained above, in some arrangements, the platform supporting motor housing 211 may be the patient, since motor housing 211 may be positioned close to the insertion point. In other embodiments, the platform supporting motor housing 211 may be an external structure.

To couple drive assembly 203 to driven assembly 201, the clinician or user may insert the proximal portion of flow diverter 205 into opening 202 in cap 212 of motor housing 211. After passing through opening 202, the proximal portion of flow diverter 205 may reside within a recess formed within motor housing 211. In some embodiments, a securement device is configured to lock or secure drive assembly 203 to driven assembly 201 once driven assembly 201 is fully inserted into drive assembly 203. In other embodiments, the securement device may be configured to secure drive assembly 203 to driven assembly 201 by inserting driven assembly 201 into drive assembly 203, and then rotating drive assembly 203 with respect to driven assembly 201. In some embodiments, coupling drive assembly 203 to driven assembly 201 may be irreversible, such that there may be no release mechanism to decouple drive assembly 203 from driven assembly 201. In embodiments without a release mechanism, catheter assembly 100A (including driven assembly 201) and motor housing 211 may be disposable components. In other embodiments, a release mechanism may be provided to remove drive assembly 203 from driven assembly 201. Drive assembly 203 may thereby be used multiple times in some embodiments.

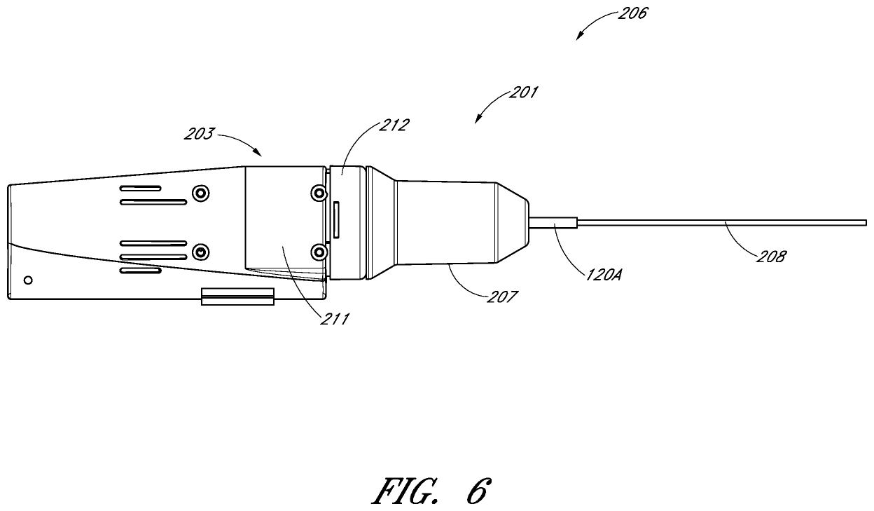

FIG. 6 illustrates motor assembly 206 in the assembled state, for example, after drive assembly 203 has been secured to driven assembly 201. When drive assembly 203 is activated (e.g., a motor is activated to rotate an output shaft), driven assembly 201, which is operably coupled to drive assembly 203, is also activated. Activated driven assembly 203 may cause drive shaft 208 to rotate, which in turn causes the impeller to rotate to thereby pump blood through the patient.

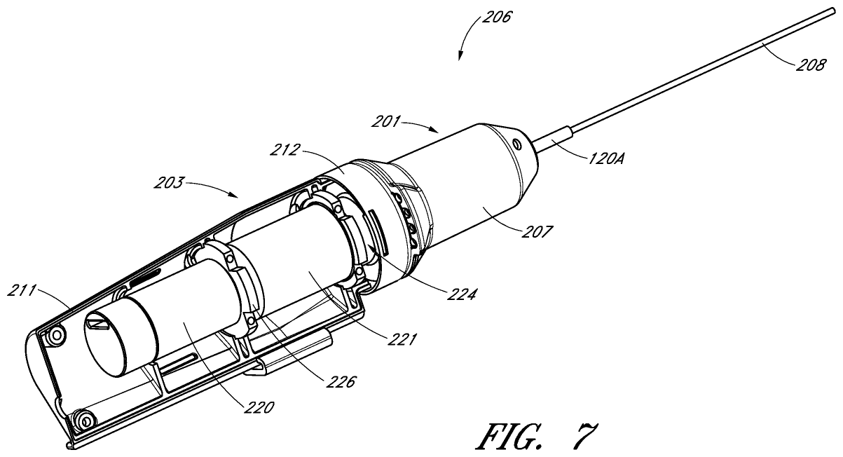

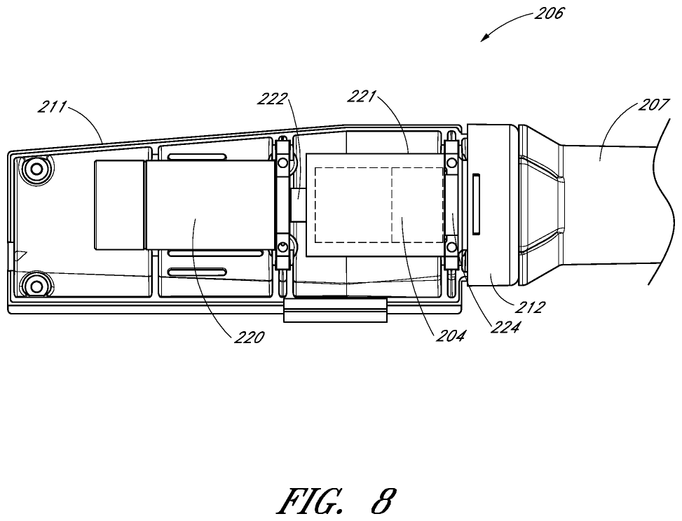

FIGS. 7 and 8 illustrate motor assembly 206 with one wall of motor housing 211 removed so that various internal components in motor housing 211 may be better illustrated. A motor 220 may be positioned within motor housing 211 and mounted by way of a motor mount 226. Motor 220 may be operably coupled to a drive magnet 221. For example, motor 220 may include an output shaft 222 that rotates drive magnet 221. In some embodiments, drive magnet 221 may rotate relative to motor mount 226 and motor housing 211. Further, in some embodiments, drive magnet 221 may be free to translate axially between motor mount 226 and a barrier 224. One advantage of the translating capability is to enable drive magnet 221 and driven magnet 204 to self-align by way of axial translation. Barrier 224 may be mounted to motor housing 211 and at least partially within cap 212 to support at least drive magnet 221. In other embodiments, drive assembly 203 may include a plurality of motor windings configured to induce rotation of drive magnet 221. In still other embodiments, the motor windings may operate directly on driven magnet 204 within driven assembly 201. For example, the motor windings may be activated in phases to create an electric field and thereby commutate driven magnet 204.

In FIG. 8, drive magnet 221 is illustrated in phantom, such that driven magnet 204 can be seen disposed within drive magnet 221. Although not illustrated, the poles of drive magnet 221 may be formed on an interior surface of drive magnet 221, and the poles of driven magnet 204 may be formed on an exterior surface of driven magnet 204. As driven magnet 204 rotates the poles of drive magnet 221 may magnetically engage with corresponding, opposite poles of driven magnet 204 to cause driven magnet 204 to rotate with, or follow, drive magnet 221. Because driven magnet 204 may be mechanically coupled to drive shaft 208, rotation of drive magnet 221 may cause driven magnet 204 and drive shaft 208 to rotate at a speed determined in part by the speed of motor 220. Furthermore, when driven magnet 204 is inserted into drive magnet 221, the poles of each magnet may cause drive magnet 221 and driven magnet 204 to self-align. The magnetic forces between drive magnet 221 and driven magnet 204 may assist in coupling drive assembly 203 to driven assembly 201.

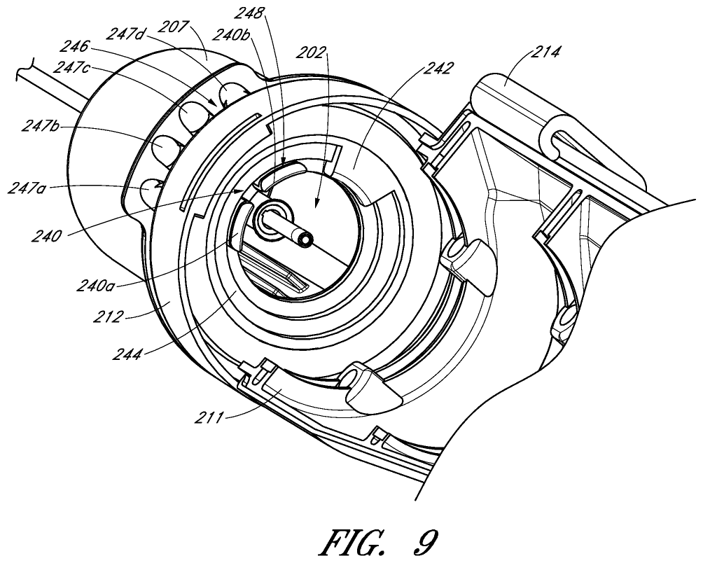

Turning to FIG. 9, a perspective view of various components at the interface between drive assembly 203 and driven assembly 201 is shown. Various components have been hidden to facilitate illustration of one means to secure drive assembly 203 to driven assembly 201. A first securement device 240 is illustrated in FIG. 9. First securement device 240 may include a first projection 240a and a second projection 240b. Furthermore, a locking recess 244 may be formed in cap 212 around at least a portion of a perimeter of opening 202. A lip 242 may also extend from the perimeter at least partially into opening 202. As shown, lip 242 may also extend proximally from locking recess 244 such that a step is formed between locking recess 244 and lip 242. Further, a flange 246 may be coupled to or formed integrally with flow diverter housing 207. Flange 246 may include a plurality of apertures 247a, 247b, 247c, 247d that are configured to permit tubes and cables to pass therethrough to fluidly communicate with lumens within flow diverter 205. In some embodiments, three tubes and one electrical cable may pass through apertures 247a-d. For example, the electrical cable may be configured to electrically couple to a sensor within catheter assembly 100A, e.g., a pressure sensor. The three tubes may be configured to carry fluid to and from catheter assembly 100A. For example, a first tube may be configured to carry infusate into catheter assembly 100A, a second tube may be configured to transport fluids to the pressure sensor region, and the third tube may be configured to transport waste fluid out of catheter assembly 100A. Although not illustrated, the tubes and cable(s) may pass through apertures 247a-d of flange 246 and may rest against motor housing 211. By organizing the routing of the tubes and cable(s), apertures 247a-d may advantageously prevent the tubes and cable(s) from becoming entangled with one another or with other components of catheter assembly 100A.

When driven assembly 201 is inserted into opening 202, first and second projections 240a, 240b may pass through the opening and engage locking recess 244. In some embodiments, projections 240a, 240b and locking recess 244 may be sized and shaped such that axial translation of projections 240a, 240b through opening 202 causes a flange or tab 248 at a distal end of each projection 240a, 240b to extend over locking recess 244. Thus, in some embodiments, once projections 240a, 240b are inserted through opening 202, tabs 248 at the distal end of projections 240a, 240b are biased to deform radially outward to engage locking recess 244 to secure driven assembly 201 to drive assembly 203.

Once driven assembly 201 is secured to drive assembly 203, flow diverter housing 207 may be rotated relative to cap 212. By permitting relative rotation between driven assembly 201 and drive assembly 203, the clinician is able to position impeller assembly 116A within the patient at a desired angle or configuration to achieve the best pumping performance. As shown in FIG. 9, lip 242 may act to restrict the relative rotation between driven assembly 201 (e.g., flow diverter housing 207) and drive assembly 203 (e.g. cap 212 and motor housing 211). As illustrated, flange 246 and apertures 247a-d may be circumferentially aligned with projections 240a, 240b. Further, lip 242 may be circumferentially aligned with sliding member 213, track 214, and connector 291 of motor housing 211. If flange 246 and projections 240a, 240b are rotated such that they circumferentially align with lip 242, then the tubes and cable(s) that extend from apertures 247a-d may become entangled with or otherwise obstructed by sliding member 213 and connector 291. Thus, it may be advantageous to ensure that sliding member 213 and connector 291 (or any other components on the outer surface of motor housing 211) do not interfere or obstruct the tubes and cable(s) extending out of apertures 247a-d of flange 246. Lip 242 formed in cap 212 may act to solve this problem by ensuring that flange 246 is circumferentially offset from sliding member 213 and connector 291. For example, flow diverter housing 207 may be rotated until one of projections 240a, 240b bears against a side of lip 242. By preventing further rotation beyond the side of lip 242, lip 242 may ensure that flange 246 and apertures 247a-d are circumferentially offset from sliding member 213, track 214, and connector 291.

In one embodiment, once catheter assembly 100A is secured to motor housing 211, the connection between driven assembly 201 and drive assembly 203 may be configured such that drive assembly 203 may not be removed from driven assembly 201. The secure connection between the two assemblies may advantageously ensure that motor housing 211 is not accidentally disengaged from catheter assembly 100A during a medical procedure. In such embodiments, both catheter assembly 100A and drive assembly 203 may preferably be disposable.

In other embodiments, it may be desirable to utilize a re-usable drive assembly 203. In such embodiments, drive assembly 203 may be removably engaged with catheter assembly 100A (e.g., engaged with driven assembly 201). For example, lip 242 may be sized and shaped such that when drive assembly 203 is rotated relative to driven assembly 201, tabs 248 are deflected radially inward over lip 242 such that driven assembly 201 may be withdrawn from opening 202. For example, lip 242 may include a ramped portion along the sides of lip 242 to urge projections 240a, 240b radially inward. It should be appreciated that other release mechanisms are possible.

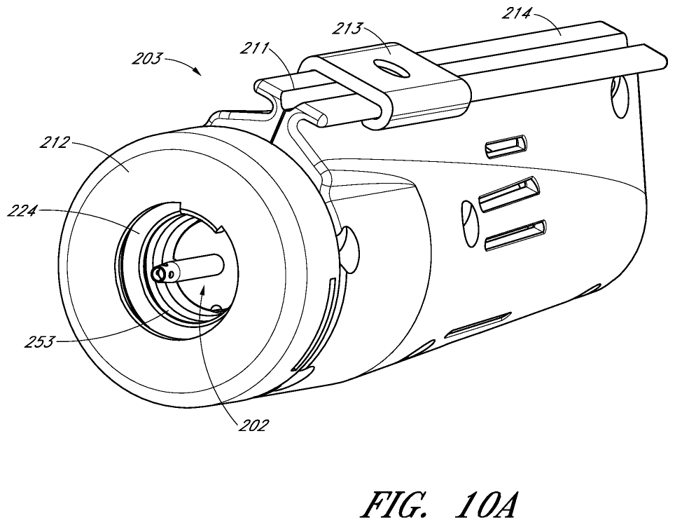

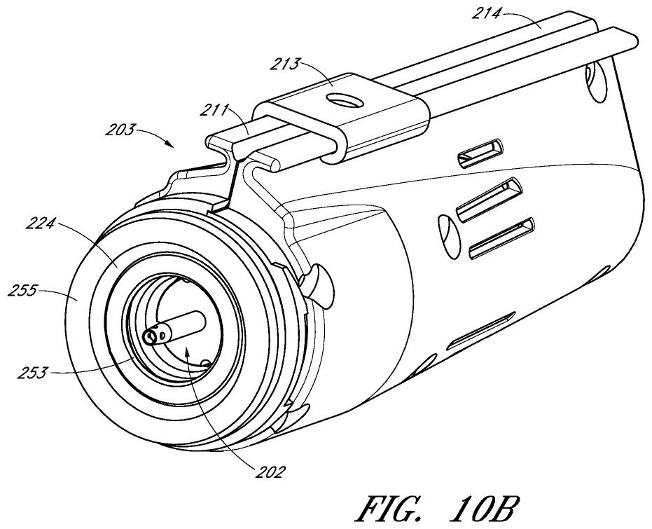

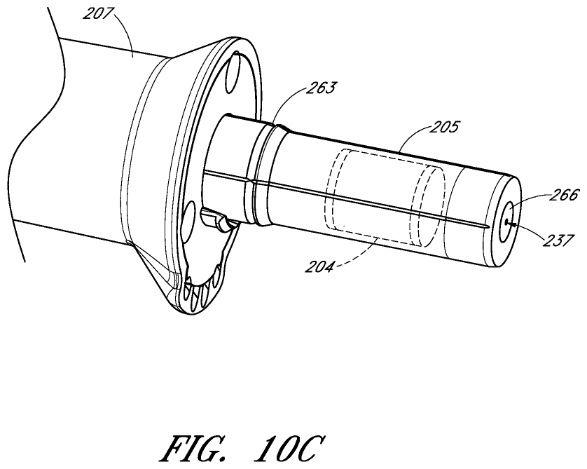

FIGS. 10A-10C illustrate an additional means to secure drive assembly 203 to driven assembly 201. As shown in the perspective view of FIG. 10A, a locking O-ring 253 may be mounted to barrier 224 that is disposed within motor housing 211 and at least partially within cap 212. In particular, locking O-ring 253 may be mounted on an inner surface of drive assembly 203 or motor housing 211 surrounding the recess or opening 202 into which driven assembly 201 may be received. As explained below, locking O-ring 253 may act as a detent mechanism and may be configured to be secured within an arcuate channel formed in an outer surface of driven assembly 201 (e.g., in an outer surface of flow diverter 205 in some embodiments). In other embodiments, various mechanisms may act as a detent to secure driven assembly 201 to drive assembly 203. For example, in one embodiment, a spring plunger or other type of spring-loaded feature may be cut or molded into barrier 224, in a manner similar to locking O-ring 253 of FIGS. 10A-10C. The spring plunger or spring-loaded feature may be configured to engage the arcuate channel, as explained below with respect to FIG. 10C. Skilled artisans will understand that other types of detent mechanisms may be employed.

FIG. 10B illustrates the same perspective of drive assembly 203 as shown in FIG. 10A, except cap 212 has been hidden to better illustrate locking O-ring 253 and a second, stabilizing O-ring 255. Stabilizing O-ring 255 is an example of a damper that may be provided between motor 220 and catheter assembly 100A. In some embodiments, the damper may provide a vibration absorbing benefit. In other embodiment, the damper may reduce noise when catheter pump 10 (shown in FIG. 1) is operating. In some embodiments, the damper may also both absorb vibration and reduce noise. Stabilizing O-ring 255 may be disposed within cap 212 and may be sized and shaped to fit along the inner recess forming the inner perimeter of cap 212. Stabilizing O-ring 255 may be configured to stabilize cap 212 and motor housing 211 against vibrations induced by operation of motor 220. For example, as motor housing 211 and/or cap 212 vibrate, stabilizing O-ring 255 may absorb the vibrations transmitted through cap 212. Stabilizing O-ring 255 may support cap 212 to prevent cap 212 from deforming or deflecting in response to vibrations. In some embodiments, stabilizing O-ring 255 may act to dampen the vibrations, which may be significant given the high rotational speeds involved in the exemplary device.

In further embodiments, a damping material may also be applied around motor 220 to further dampen vibrations. The damping material may be any suitable damping material (e.g., a visco-elastic or elastic polymer). For example, the damping material may be applied between motor mount 226 and motor 220. In addition, the damping material may also be applied around the body of motor 220 between motor 220 and motor housing 211. In some embodiments, the damping material may be captured by a rib formed in motor housing 211. In some embodiments, the rib may be formed around motor 220.

Turning to FIG. 10C, a proximal end of driven assembly 201 is shown. As explained above, flow diverter 205 (or the housing of flow diverter 205 in some embodiments) can include an arcuate channel 263 formed in an outer surface of flow diverter 205. Arcuate channel 263 may be sized and shaped to receive locking O-ring 253 when flow diverter 205 is inserted into opening 202 of drive assembly 203. As flow diverter 205 is axially translated through the recess or opening 202, locking O-ring 253 may be urged or slid over an edge of arcuate channel 263 and may be retained in arcuate channel 263. Thus, locking O-ring 253 and arcuate channel 263 may operate to act as a second securement device. Axial forces applied to motor assembly 206 may thereby be mechanically resisted, as the walls of arcuate channel 263 bear against locking O-ring 253 to prevent locking O-ring 253 from translating relative to arcuate channel 263. In various embodiments, other internal locking mechanisms (e.g., within driven assembly 201 and/or drive assembly 203) may be provided to secure driven and drive assemblies 201, 203 together. For example, driven magnet 204 and drive magnet 221 may be configured to assist in securing the two assemblies together, in addition to aligning the poles of the magnets. Other internal locking mechanisms may be suitable.

FIG. 10C also illustrates a resealable member 266 disposed within the proximal end portion of driven assembly 201 (e.g., the proximal end of catheter assembly 100A as shown in FIG. 4). As in FIG. 4, proximal guidewire opening 237 may be formed in resealable member 266. As explained above with respect to FIG. 4, guidewire 235 may be inserted through proximal guidewire opening 237 and may be maneuvered through the patient's vasculature. After guiding the operative device of catheter pump 10 (shown in FIG. 1) to the heart, guidewire 235 may be removed from catheter assembly 100A by pulling guidewire 235 out through proximal guidewire opening 237. Because fluid may be introduced into flow diverter 205, it may be advantageous to seal the proximal end of flow diverter 205 to prevent fluid from leaking out of catheter assembly 100A. Resealable member 266 may be formed of an elastic, self-sealing material that is capable of closing and sealing proximal guidewire opening 237 when guidewire 235 is removed. Resealable member 266 may be formed of any suitable material, such as an elastomeric material. In some embodiments, resealable member 266 may be formed of any suitable polymer (e.g., a silicone or polyisoprene polymer). Skilled artisans will understand that other suitable materials may be used.

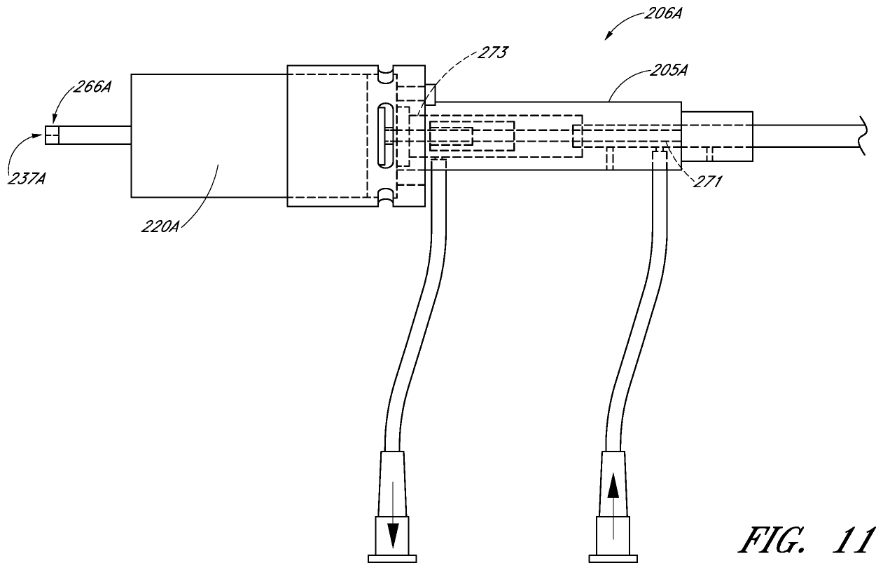

FIG. 11 illustrates yet another embodiment of a motor assembly 206A coupled to a catheter assembly, such as catheter assembly 100A (shown in FIG. 4). In FIG. 11, a flow diverter is disposed over and coupled to a catheter body 271 that may include a multi-lumen sheath configured to transport fluids into and away from the catheter assembly. Flow diverter 205A may provide support to catheter body 271 and a drive shaft configured to drive an impeller assembly, such as impeller assembly 92 (shown in FIGS. 2 and 3) and/or impeller assembly 116A (shown in FIG. 4). Further, motor assembly 206A can include a motor 220A that has a hollow lumen therethrough. Unlike the embodiments disclosed in FIGS. 4-10C, guidewire 235 may extend through proximal guidewire opening 237A formed proximal to motor 220A, rather than between motor 220A and flow diverter 205A. A resealable member 266A may be formed in proximal guidewire opening 237A such that resealable member 266A may close opening 237A when guidewire 235 is removed from the catheter assembly. A rotary seal 273 may be disposed inside a lip of flow diverter 205A. Rotary seal 273 may be disposed over and may contact a motor shaft extending from motor 220A. Rotary seal 273 may act to seal fluid within flow diverter 205A. In some embodiments, a hydrodynamic seal maybe created to prevent fluid from breaching rotary seal 273.

In the embodiment shown in FIG. 11, motor 220A may be permanently secured to flow diverter 205A and the catheter assembly. Because proximal guidewire opening 237 is positioned proximal to motor 220A, motor 220A need not be coupled with the catheter assembly in a separate coupling step. In this embodiment, motor 220A and the catheter assembly may be disposable. Motor 220A may include an output shaft and rotor magnetically coupled with a rotatable magnet in flow diverter 205A. Motor 220A may also include a plurality of windings that are energized to directly drive the rotatable magnet in flow diverter 205A.

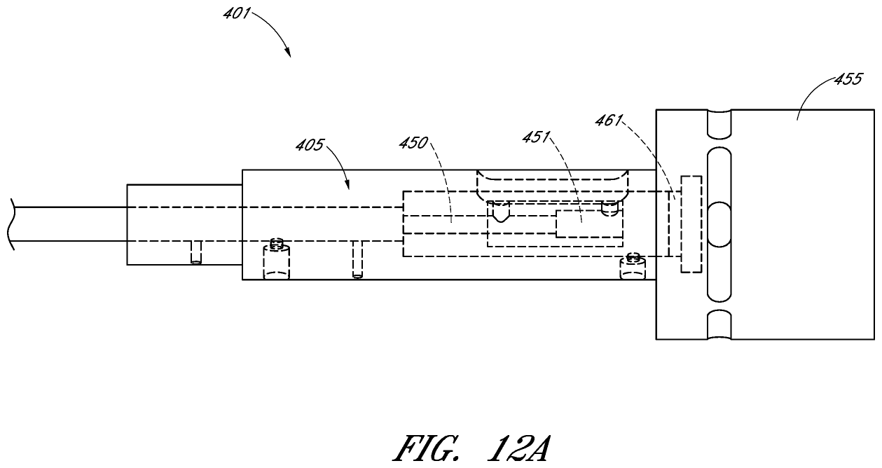

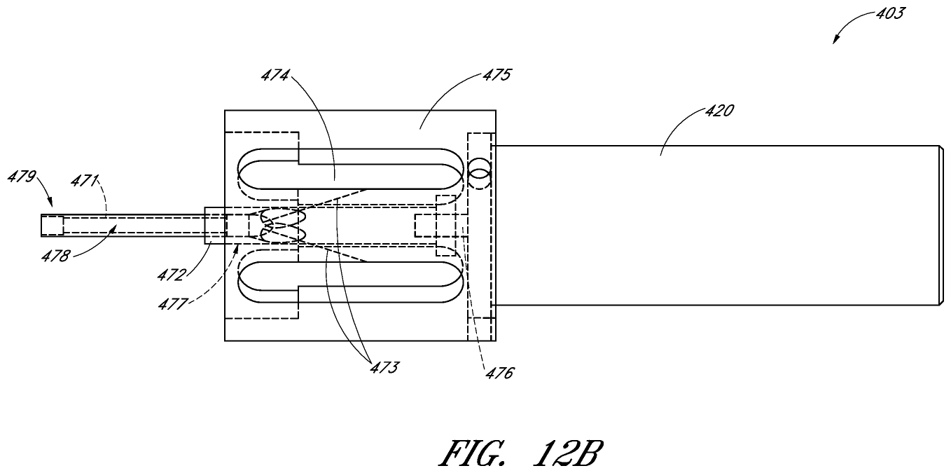

FIGS. 12A and 12B illustrate another embodiment of a motor 420 coupling having a driven assembly 401 and a drive assembly 403. Unlike the implementations disclosed in FIGS. 4-10C, the embodiment of FIGS. 12A and 12B may include a mechanical coupling disposed between an output shaft of motor 420 and a proximal end of a flexible drive shaft or cable. Unlike the implementations disclosed in FIG. 11, the embodiment of FIGS. 12A and 12B may include a guidewire guide (GWG) tube that terminates at a location distal to a motor shaft 476 that extends from motor 420. As best shown in FIG. 12B, an adapter shaft 472 may be operably coupled to motor shaft 476 extending from motor 420. A distal end portion 477 of adapter shaft 472 may be mechanically coupled to a proximal portion of an extension shaft 471 having a central lumen 478 therethrough. As shown in FIG. 12B, one or more trajectories 473 may be formed in channels within a motor housing 475 at an angle to central lumen 478 of extension shaft 471. Motor housing 475 may enclose at least adapter shaft 472 and may include one or more slots 474 formed through a wall of housing 475.

In some embodiments, a guidewire (not shown in FIG. 12B) may pass through the GWG tube from the distal end portion of the catheter assembly and may exit the catheter assembly through central lumen 478 near distal end portion 477 of adapter shaft 472 (or, alternatively, near the proximal end portion of extension shaft 471). In some embodiments, one of extension shaft 471 and adapter shaft 472 may include a resealable member disposed therein to reseal central lumen 478 through which the guidewire passes, as explained above. In some embodiments, extension shaft 471 and adapter shaft 472 may be combined into a single structure. When the guidewire exits central lumen 478, the guidewire may pass along angled trajectories 473 which may be formed in channels and may further pass through slots 474 to the outside environs. Trajectories 473 may follow from angled ports in adapter shaft 472. A clinician may thereby pull the guidewire through slots 474 such that the end of the guidewire may easily be pulled from the patient after guiding the catheter assembly to the heart chamber or other desired location. Because the guidewire may extend out the side of motor housing 475 through slots 474, motor shaft 476 and motor 420 need not include a central lumen for housing the guidewire. Rather, motor shaft 476 may be solid and the guidewire may simply pass through slots 474 formed in the side of motor housing 475.

Furthermore, drive assembly 403 may be mechanically coupled to driven assembly 401. For example, a distal end portion 479 of extension shaft 471 may be inserted into an opening in a flow diverter housing 455. Distal end portion 479 of extension shaft 471 may be positioned within a recess 451 and may couple to a proximal end of a drive cable 450 that is mechanically coupled to the impeller assembly. A rotary seal 461 may be positioned around the opening and may be configured to seal motor 420 and/or motor housing 475 from fluid within flow diverter 405.

Advantageously, the embodiments of FIGS. 12A and 12B enable motor 420 to be positioned proximal of rotary seal 461 in order to minimize or prevent exposing motor 420 to fluid that may inadvertently leak from flow diverter 405. It should be appreciated that extension shaft 471 may be lengthened in order to further isolate or separate motor 420 from fluid diverter 405 in order to minimize the risk of leaking fluids.

FIG. 13 illustrates further features that may be included in various embodiments. In particular, FIG. 13 illustrates a distal end portion 300 of a catheter assembly, such as catheter assembly 100A (shown in FIG. 4). As shown a cannula housing 302 may be coupled to a distal tip member 304. Distal tip member 304 may be configured to assist in guiding the operative device of the catheter assembly (e.g., an impeller assembly, which may be similar to or the same as impeller assembly 116A shown in FIG. 4), along guidewire 235. The exemplary distal tip member 304 is formed of a flexible material and has a rounded end to prevent injury to the surrounding tissue. If distal tip member 304 contacts a portion of the patient's anatomy (such as a heart wall or an arterial wall), distal tip member 304 will safely deform or bend without harming the patient. Distal tip member 304 may also serve to space the operative device away from the tissue wall. In addition, a guide feature or guidewire guide (GWG) tube 312 may be provided. GWG tube 312, discussed above with reference to FIG. 4, may extend through a central lumen (such as central lumen 478 shown in FIG. 12B) of the catheter assembly. Thus, GWG tube 312 may pass through the impeller shaft (not shown in FIG. 13, as the impeller is located proximal to distal end portion 300) and a lumen formed within distal tip member 304. GWG tube 312 may include the central lumen extending throughout the length of the catheter assembly. In the embodiment of FIG. 13, GWG tube 312 may extend distally past the distal end of distal tip member 304. As explained above, in various embodiments, the clinician may introduce a proximal end of guidewire 235 into the distal end of GWG tube 312, which in FIG. 13 extends distally beyond distal tip member 304. In some embodiments, once guidewire 235 has been inserted into the patient, GWG tube 312 may be removed from the catheter assembly.

Distal tip member 304 may include a flexible, central body 306, a proximal coupling member 308, and a rounded tip 310 at the distal end of distal tip member 304. Central body 306 may provide structural support for distal tip member 304. Proximal coupling member 308 may be coupled to or integrally formed with central body 306. Proximal coupling member 308 may be configured to couple the distal end of cannula housing 302 to distal tip member 304. Rounded tip 310, also referred to as a ball tip, may be integrally formed with central body 306 at the distal end of distal tip member 304. Because rounded tip 310 is flexible and has a round shape, if distal tip member 304 contacts or interacts with the patient's anatomy, rounded tip 310 may have sufficient compliance so as to deflect away from the anatomy instead of puncturing or otherwise injuring the anatomy. As compared with other embodiments, distal tip member 304 may advantageously include sufficient structure by way of central body 306 such that distal tip member 304 may accurately track guidewire 235 to position the impeller assembly within the heart. Yet, because distal tip member 304 is made of a flexible material and includes rounded tip 310, any mechanical interactions with the anatomy may be clinically safe for the patient.

One potential problem with the embodiment of FIG. 13 is that it may be difficult for the clinician to insert guidewire 235 into the narrow lumen of GWG tube 312. Since GWG tube 312 has a small inner diameter relative to the size of the clinician's hands, the clinician may have trouble inserting guidewire 235 into the distal end of GWG tube 312, which extends past the distal end of distal tip member 304. In addition, when the clinician inserts guidewire 235 into GWG tube 312, the distal edges of GWG tube 312 may scratch or partially remove a protective coating applied on the exterior surface of guidewire 235. Damage to the coating on guidewire 235 may harm the patient as the partially uncoated guidewire 235 is passed through the patient's vasculature. Accordingly, it may be desirable in various arrangements to make it easier for the clinician to insert guidewire 235 into distal end portion 300 of the catheter assembly, and/or to permit insertion of guidewire 235 into the catheter assembly while maintaining the protective coating on guidewire 235.

Additionally, as explained herein, in some embodiments, cannula housing 302 (which may form part of an operative device) may be collapsed into a stored configuration such that cannula housing 302 is disposed within an outer sheath. When cannula housing 302 is disposed within the outer sheath, a distal end or edge of the outer sheath may abut distal tip member 304. In some cases, the distal edge of the outer sheath may extend over distal tip member 304, or the sheath may have an outer diameter such that the distal edge of the outer sheath is exposed. When the sheath is advanced through the patient's vasculature, the distal edge of the outer sheath may scratch, scrape, or otherwise harm the anatomy. Accordingly, there is a need to prevent harm to the patient's anatomy due to scraping of the distal edge of the sheath against the vasculature.

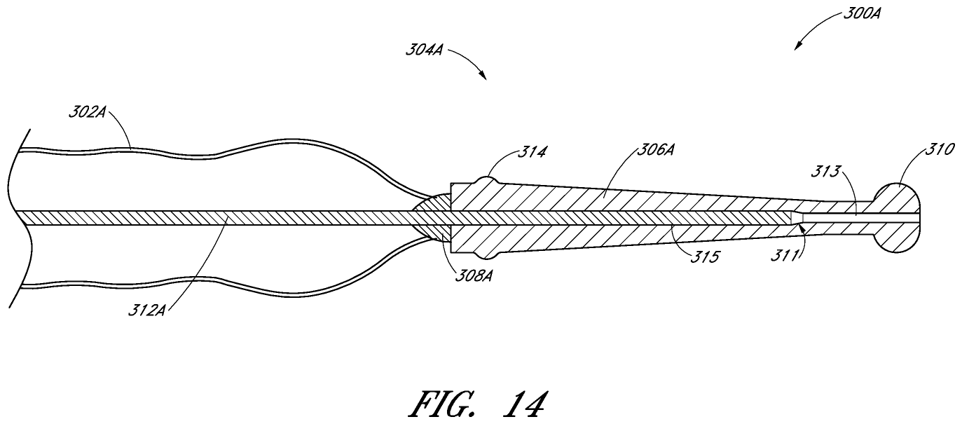

FIG. 14 is a side cross-sectional view of a distal tip member 304A disposed at a distal end 300A of the catheter assembly, according to another embodiment. Unless otherwise noted, the reference numerals in FIG. 14 may refer to components similar to or the same as those in FIG. 13. For example, as with FIG. 13, distal tip member 304A may be coupled to a cannula housing 302A. Distal tip member 304A may include a flexible, central body 306A, a proximal coupling member 308A, and a rounded tip 310A at distal end of distal tip member 304A. Furthermore, as with FIG. 13, a guide feature or guidewire guide tube (GWG) 312A may pass through cannula housing 302A and a lumen passing through distal tip member 304A.

However, unlike the embodiment of FIG. 13, central body 306A may include a bump 314 disposed near a proximal portion of distal tip member 304A. Bump 314 illustrated may advantageously prevent the outer sheath from scraping or scratching the anatomy when the sheath is advanced through the patient's vascular system. For example, when cannula housing 302A is disposed within the outer sheath, the sheath will advance over cannula housing 302A such that the distal edge or end of the sheath will abut or be adjacent bump 314 of distal tip member 304A. Bump 314 may act to shield the patient's anatomy from sharp edges of the outer sheath as distal end 300A is advanced through the patient. Further, the patient may not be harmed when bump 314 interact with the anatomy because bump 314 includes a rounded, smooth profile. Accordingly, bump 314 may advantageously improve patient outcomes by further protecting the patient's anatomy.

Furthermore, GWG tube 312A does not extend distally past the end of distal tip member 304A. Rather, in FIG. 14, the central lumen passing through distal tip member 304A may include a proximal lumen 315 and a distal lumen 313. As shown in FIG. 14, proximal lumen 315 may have an inner diameter larger than an inner diameter of distal lumen 313. A stepped portion or shoulder 311 may define the transition between proximal lumen 315 and distal lumen 313. As illustrated in FIG. 14, the inner diameter of proximal lumen 315 is sized to accommodate GWG tube 312A as it passes through a portion of distal tip member 304A. However, the inner diameter of distal lumen 313 is sized to be smaller than the outer diameter of GWG tube 312A such that GWG tube 312A is too large to pass through distal lumen 313 of distal tip member 304A. In addition, in some embodiments, the thickness of GWG tube 312A may be made smaller than the height of the stepped portion or shoulder 311 (e.g., smaller than the difference between the inner diameter of proximal lumen 315 and the inner diameter of distal lumen 313). By housing GWG tube 312A against shoulder 311, shoulder 311 may protect the outer coating of a guidewire (such as guidewire 235 shown in FIG. 4) when the guidewire is inserted proximally from distal lumen 313 to proximal lumen 315.

The embodiment illustrated in FIG. 14 may assist the clinician in inserting the guidewire into distal end 300A of the catheter assembly. For example, GWG tube 312A may be inserted through the central lumen of the catheter assembly. For example, GWG tube 312A may pass distally through a portion of the motor, the catheter body, the impeller assembly, and cannula housing 302A, and through proximal lumen 315 of distal tip member 304A. GWG tube 312A may be urged further distally until the distal end of GWG tube 312A reaches shoulder 311. When the distal end of GWG tube 312A reaches shoulder 311, shoulder 311 may prevent further insertion of GWG tube 312 in the distal direction (e.g., shoulder 311 may have a smaller diameter that the diameter of GWG tube 312A). Because the inner diameter of distal lumen 313 is smaller than the outer diameter of GWG tube 312A, the distal end of GWG tube 312A may be disposed just proximal of shoulder 311, as shown in FIG. 14. Shoulder 311 may be made of a flexible material, which may result in expansion of shoulder 311 when a distal end of GWG tube 312A is pushed against shoulder 311. Therefore, in some embodiments, shoulder 311 may be coupled to a rigid ring (not shown) that forms a non-deformable ledge. The ring facilitates maintaining the diameter of shoulder 311 and prevents GWG 312A from expanding shoulder 311 and moving beyond shoulder 311.

The clinician may insert the proximal end of the guidewire proximally through distal lumen 313 passing through rounded tip 310A at the distal end of distal tip member 304A. Because distal tip member 304A is flexible, the clinician may easily bend or otherwise manipulate the distal end of distal tip member 304A to accommodate the small guidewire. Unlike GWG tube 312A, which may be generally stiffer than distal tip member 304A, the clinician may easily deform distal tip member 304A to urge the guidewire into distal lumen 313. Once the guidewire is inserted in distal lumen 313, the clinician may urge the guidewire proximally past stepped portion or shoulder 311 and into a larger GWG tube 312A, which may be positioned within proximal lumen 315. Furthermore, since most commercial guidewires include a coating (e.g. a hydrophilic or antimicrobial coating, or PTFE coating), GWG tube 312A and shoulder 311 advantageously avoid damaging or removing the coating. When the wall thickness of GWG tube 312A is less than the height of the step or shoulder 311, shoulder 311 may substantially prevent GWG tube 312A from scraping the exterior coating off of the guidewire. Instead, the guidewire easily passes from distal lumen 313 to proximal lumen 315. The guidewire may then be urged proximally through the impeller and catheter assembly until the guidewire protrudes from the proximal end of the system, such as through proximal guidewire opening 237 described above with reference to FIG. 4.

The GWG features (e.g., GWG tubes 312, 312A) illustrated in FIGS. 13 and 14 include a central lumen passing through the catheter assembly along its length. In some embodiments, it may be desirable to omit the central lumen through the catheter assembly. For example, removing the central lumen from the drive cable and motor assembly may advantageously simplify the manufacturing process and may reduce the profile (e.g., diameter) of the catheter assembly.

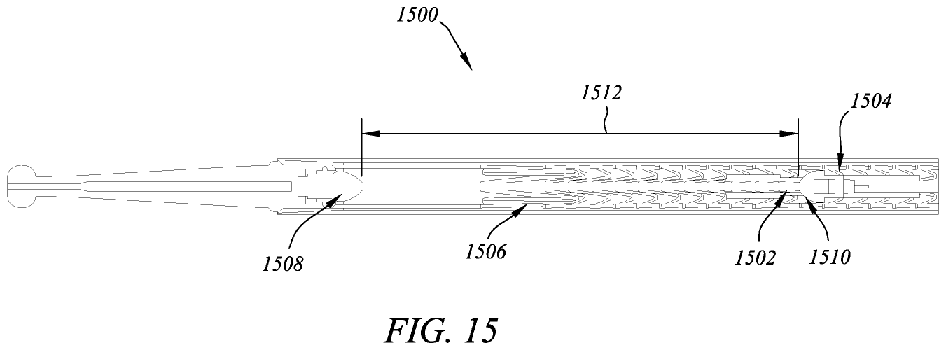

FIG. 15 is a side cross-sectional view of a catheter assembly 1500, such as catheter assembly 100A (shown in FIG. 4). Catheter assembly 1500 includes an embodiment of a guidewire guide (GWG) tube 1502, similar to GWG tubes 312 and 312A (shown in in FIGS. 13 and 14). Catheter assembly 1500 also includes a distal septum 1504, an expandable cannula 1506 (shown in a collapsed state in FIG. 15), a flexible atraumatic tip (FAT) 1508, a distal end 1510 of an impeller, and a gap 1512 extending between FAT 1508 a distal end 1510. GWG tube 1502 extends across gap 1512 and extends through cannula 1506. GWG 1502 also extends through distal end 1510 and across distal septum 1504.

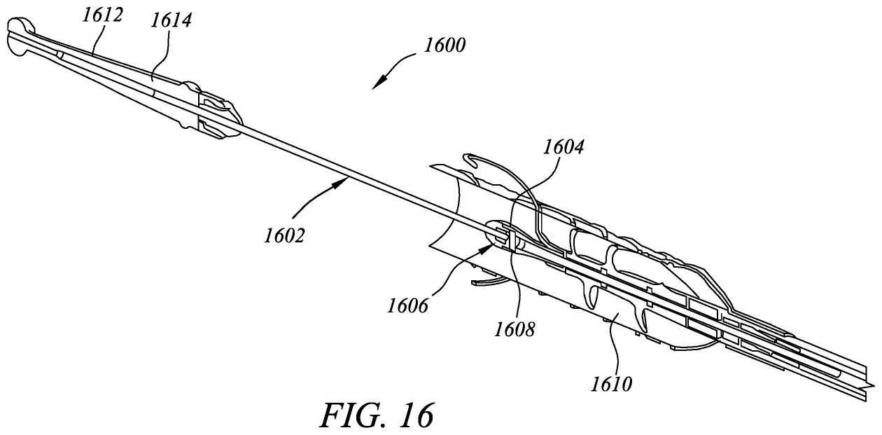

FIG. 16 is a perspective cross-sectional view of an alternative catheter assembly 1600, such as catheter assembly 100A (shown in FIG. 4). Catheter assembly 1600 includes a guidewire guide (GWG) 1602, a proximal end 1604 of GWG 1602, an impeller tip 1606, a distal septum 1608, a cannula 1610, a flexible atraumatic tip (FAT) 1612, similar to FAT 1508 (shown in FIG. 15), and a distal end 1614 of GWG 1602. In this embodiment, GWG 1602 is permanently coupled between FAT 1612 and impeller tip 1606 (e.g., GWG 1602 remains in catheter assembly 1600 prior to a procedure, such as a percutaneous heart procedure, and during the procedure). GWG 1602 is flexible and resilient to facilitate (a) not kinking as catheter assembly 1600 is delivered to the left ventricle of a patient's heart and (b) not increasing a rigid length of catheter assembly 1600.

GWG 1602 may be made of flexible alloys, such as nitinol, a grade of stainless steel, and/or advanced polymers such as polyamide, HDPE, LLDPE, FEP, PET, Pebax, etc. Any of these materials may include a pattern cut into them to improve flexibility and prevent kinking. GWG 1602 may also include a reinforced sheath (e.g., a thin braid structure) that also improves flexibility and prevents kinking (particularly in embodiments where GWG 1602 rotates during operation of catheter assembly 1600).

As shown in FIG. 16, proximal end 1604 of GWG 1602 is located distal of distal septum 1608. Accordingly, GWG 1602 does not extend across distal septum 1608. Accordingly, by permanently including GWG 1602 in catheter assembly 1600, the only time distal septum 1608 is pierced is when a user (e.g., a clinician) passes a guidewire through GWG 1602 prior to a procedure. The guidewire may subsequently be removed, for example, as soon as catheter assembly 1600 is delivered inside the patient, thereby allowing distal septum 1608 to be sealed quickly (e.g., in less than one hour).

As cannula 1610 is sheathed, FAT 1612 moves distally by approximately 0.25 inches relative to impeller tip 1606. To accommodate this, in the embodiment shown in FIG. 16, distal end 1614 of GWG 1602 is coupled to FAT 1612 using a slip fit. Further, in the embodiment shown, GWG 1602 is coupled to FAT 1612, such that GWG 1602 is capable of rotating or spinning relative to FAT 1612 (and rotating with the impeller) during operation of catheter assembly 1600. In other embodiments, where GWG 1602 is not rotatable or spinnable with the impeller, at least one bearing (not shown) is included in catheter assembly 1600, such that the at least one bearing is configured to secure GWG 1602 inside catheter assembly 1600 and allow the impeller to rotate relative to GWG 1602.

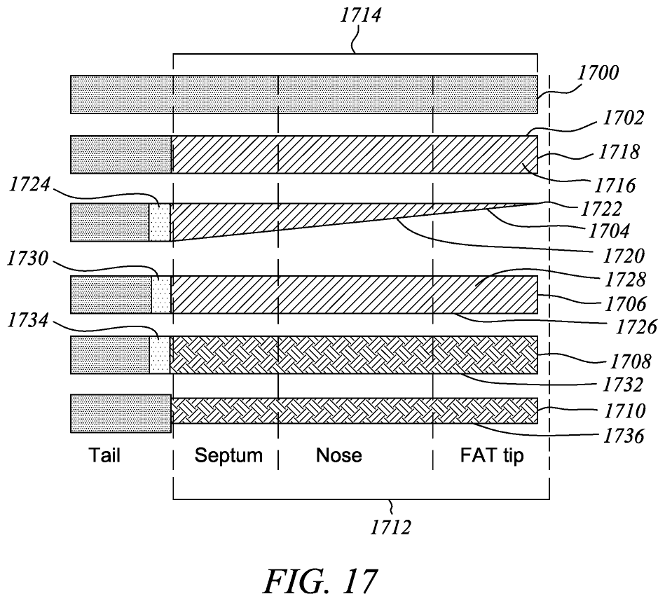

FIG. 17 illustrates various embodiments of a distal portion of a GWG tube (also referred to herein as a hypotube) that may be used to implement, for example, GWG tubes 312 and 312A (shown in FIGS. 13 and 14).

A first hypotube 1700 shown in FIG. 16 represents a known configuration of a GWG tube. First hypotube 1700 may include a relatively rigid hypodermic tubing made of stainless steel. Hypotubes 1702, 1704, 1706, 1708, and 1710 are alternative embodiments of hypotube 1700 with generally the same cut length as hypotube 1700. However, a distal tip 1712 of each hypotube 1702, 1704, 1706, 1708, and 1710 is different than distal tip 1714 of hypotube 1700.

Each distal tip 1712 includes a polymeric sleeve or flexible tubular lumen, thereby making the distal end of the GWG tube (which contacts the inner lumen of the sealing septum and/or rigid transition, as shown in FIG. 16) more conformable and expandable.

Second hypotube 1702 includes a sleeve 1716 (e.g., a polymeric sleeve or flexible tubular lumen) that may be extruded or constructed from any of a number of polymeric materials similar to a tip of a balloon, such as polyamide elastomers (similar to inner members of balloon catheters), polyethylene copolymers, PTFE blends, or combination thereof. Sleeve 1716 may be compliant, expandable, electrospun, braided, mesh-like, webbing-like, and/or similar to a wrap or tubular lumen. Sleeve 1716 may also be coated to further reduce friction between contact components. The coating may include silicone or mineral oil based materials, hydrophobic materials, hydrophilic materials, or other materials suitable for coating sleeve 1716 to function, as described herein. Further, in some embodiments, sleeve 1716 includes an end terminus 1718 that may include an oval or circular lumen that may expand during a guidewire insertion, for example, through the GWG tube. Sleeve 1716 may also be straight-cut or angled to reduce the total surface area of material around the guidewire, potentially that would enlarge the distal septum during storage post-manufacturing prior to use.

Third hypotube 1704 includes a lased sleeve 1720 that may be constructed from a flexible alloy, such as nitinol or a grade of stainless steel (with exemplary compositions of 16% to 25% chromium and between 6% to 25% nickel). Lased sleeve 1720 may be cut with different patterns (e.g., including the one shown in FIG. 17) and may include a coating for reduced friction. The coating may include similar materials to those described above with respect to sleeve 1716. Lased sleeve 1720 may include an end terminus 1722 that employs a cylindrical collar to ensure that no snagging on other components occurs. Lased sleeve 1720 may include relatively small outer diameter at end terminus 1722, and segments having different expansion capabilities. Lased sleeve 1720 may also include a lased transition 1724.

GWG tube may, in some embodiments, include combinations of expandable sleeves and lased transitions. For example, fourth hypotube 1706 includes a sleeve 1726 having both an expandable braided sleeve 1728 and a lased transition section 1730.

Fifth hypotube 1708 includes a lased sleeve 1732 and a lased transition 1734. Sixth hypotube 1710 includes a lased expandable sleeve 1736. Each hypotube 1702, 1704, 1706, 1708, and 1710, described above, is more “septum friendly” than at least some known GWG tubes, since each of the hypotubes has a distal tip 1712 that is more flexible and/or has a reduced footprint (e.g., smaller outer diameter), reducing friction between the hypotube and other components (e.g. tail, nose, distal septum 1504, cannula 1506, FAT 1508, distal end 1510 of the impeller (all shown in FIG. 15), and other components that may be in contact with the hypotubes). With distal tip 1712 capable of expanding only temporarily or a minimal amount of time (e.g., for less than thirty seconds during guidewire insertion by the physician), septum sealing efficiency is maintained and a material “memory” of the septum would not be impacted.

Additionally, each hypotube 1702, 1704, 1706, 1708, and 1710 may include diameters or gauge sizes smaller or larger than existing GWG tube outer diameters and/or inner diameters, in order to accommodate guidewires of varied dimensions dependent on coronary, peripheral, neurovascular, biliary, or alternate anatomies. For example, the dimensions of hypotubes 1702, 1704, 1706, 1708, and 1710 may include an outer diameter ranging from 0.0120-0.0125 inches, a wall thickness of approximately 0.002 inches, and an inner diameter ranging from 0.0075-0.0090 inches.

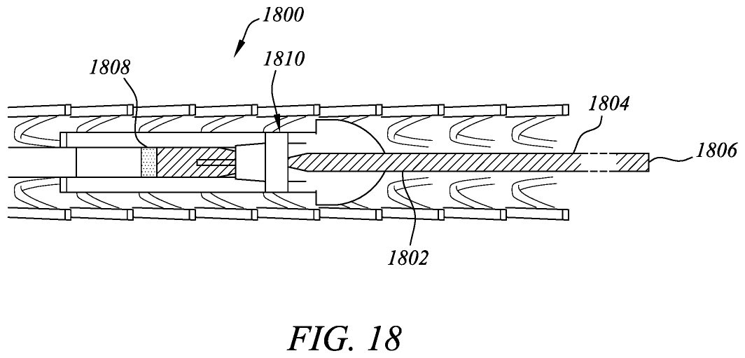

FIG. 18 is an enlarged view of a portion of catheter assembly 1800, such as catheter assembly 1500 (shown in FIG. 15). Catheter assembly 1800 includes a hypotube 1802, such as of one of the hypotubes shown in FIG. 17. Hypotube 1802 includes a lased sleeve 1804 that may be constructed from a flexible alloy, such as nitinol, a grade of stainless steel, or advanced polymers, such as polyamide, HDPE, LLDPE, FEP, PET, Pebax, etc. Lased sleeve 1804 may be cut with different patterns (e.g., including the patterns shown in FIG. 17) and may include a coating for reduced friction. The coating may include similar materials to those described above with respect to the sleeve described in FIG. 17.

As shown in FIG. 18, the diameter of lased sleeve 1804 decreases across a septum 1810, allowing septum 1810 to substantially close. Once lased sleeve 1804 exits septum 1810 distally, lased sleeve 1804 expands back out to a larger diameter (which may be the same as or smaller than a diameter of portions of the associated hypotube that are proximal of septum 1810).

The embodiments described herein provide systems and methods for guidewire guides in implantable medical devices. Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A guidewire guide (GWG) for use in a percutaneous heart pump, the GWG comprising:
a hypotube; and
a sleeve section coupled to a distal end of the hypotube, wherein the sleeve section is configured to extend across a distal septum of the percutaneous heart pump when the GWG is inserted into the percutaneous heart pump, and wherein the sleeve section facilitates reducing deformation of the distal septum while the sleeve section extends across the distal septum.

2. The GWG of claim 1, wherein the sleeve section is constructed from one or more polymeric materials.

3. The GWG of claim 1, wherein the sleeve section is coated with compositions or combinations of silicone, oil-based lubricious coatings, hydrophobic films, or hydrophilic materials, thereby reducing friction of the sleeve section with other components.

4. The GWG of claim 1, wherein the sleeve section is constructed from a flexible alloy.

5. The GWG of claim 1, wherein the sleeve section comprises an end terminus comprising an oval lumen or a circular lumen expandable during a guidewire insertion through the GWG.

6. The GWG of claim 1, wherein the sleeve section comprises a lased transition.

7. The GWG of claim 1, wherein the sleeve section comprises an expandable braided sleeve.

8. The GWG of claim 1 further comprising a distal tip that is configured to expand for at least less than thirty seconds when a guidewire is inserted through the GWG.

9. A catheter assembly for use in a percutaneous heart pump, the catheter assembly comprising:
an impeller assembly comprising:
an impeller;
an impeller tip positioned distal of the impeller; and
a distal septum positioned between the impeller and the impeller tip;
a flexible atraumatic tip (FAT) positioned distal of the impeller assembly; and
a guidewire guide (GWG) coupled between the impeller assembly and the flexible atraumatic tip, wherein a proximal end of the guidewire guide is positioned distal of the distal septum.

10. The catheter assembly of claim 9, wherein the GWG is flexible and resilient to facilitate (a) reducing kinking as the catheter assembly is delivered to a left ventricle of a patient's heart and (b) maintaining a rigid length of the catheter assembly.

11. The catheter assembly of claim 9, wherein the GWG is constructed from flexible alloys including at least one of nitinol, a grade of stainless steel, or advanced polymers such as polyamide, HDPE, LLDPE, FEP, PET, Pebax, etc.

12. The catheter assembly of claim 9, wherein the distal septum is pierceable when a guidewire passes through the GWG prior to a procedure.

13. The catheter assembly of claim 9, wherein the distal septum is configured to seal after a guidewire is removed.

14. The catheter assembly of claim 9 further comprising an expandable cannula.

15. A catheter assembly for use in a percutaneous heart pump, the catheter assembly comprising a guidewire guide (GWG) comprising:
a hypotube; and
a sleeve section coupled to a distal end of the hypotube, wherein the sleeve section is configured to extend across a distal septum of the percutaneous heart pump when the GWG is inserted into the percutaneous heart pump, and wherein the sleeve section facilitates reducing deformation of the distal septum while the sleeve section extends across the distal septum.

16. The catheter assembly of claim 15, wherein the sleeve section is constructed from one or more polymeric materials.

17. The catheter assembly of claim 15, wherein the sleeve section is fully or partially coated or lined with silicone or oil-based lubricants, conformal coatings, hydrophobic materials, or hydrophilic materials, thereby reducing friction of the sleeve section with other components.

18. The catheter assembly of claim 15, wherein the sleeve section comprises an end terminus comprising an oval lumen or a circular lumen expandable during a guidewire insertion through the GWG.

19. The catheter assembly of claim 15, wherein the sleeve section comprises at least one of a lased transition and an expandable braided sleeve.

20. The catheter assembly of claim 15, wherein the GWG further comprises a distal tip that is configured to expand for at least less than thirty seconds when a guidewire is inserted through the GWG.
